(19) United States
(12) Patent Application Publication
(10) Pub. No.: US 2015/0320507 A1
(43) Pub. Date: Nov. 12, 2015

(54) PATH CREATION USING MEDICAL IMAGING FOR PLANNING DEVICE INSERTION

(71) Applicant: Siemens Aktiengesellschaft, Munich (DE)

(72) Inventors: Feng Qiu, Plainsboro, NJ (US); Daphne Yu, Yardley, PA (US)

(73) Assignee: Siemens Aktiengesellschaft, Munich (DE)

(21) Appl. No.: 14/273,737

(22) Filed: May 9, 2014

(51) Int. Cl. A61B 19/00; G06T 19/00

(52) U.S. Cl. CPC A61B 19/50; G06T 19/003; A61B 19/56; A61B 19/5244; G06T 2210/41; A61B 2019/507; A61B 2019/505; A61B 2019/5295

(57) ABSTRACT

Efficient path planning of a medical device for insertion into a patient using medical imaging is provided. An image rendered from different transfer functions is used. One transfer function provides context, such as rendering to show a skin surface or other patient anatomical system. Another transfer function indicates local vessel, fluid, or other information for selecting points along the path. The user may select the transfer function used at different locations as well as the depth of a clipping plane at the different locations to allow unambiguous selection of a path point in the three-dimensional rendering.

### BACKGROUND

The present embodiments relate to path creation in medical planning. In particular, medical data representing a patient is rendered in order for the user to define a path of insertion within the patient for a medical device.

A device insertion path overlay on a medical image provides valuable interventional guidance for complex procedures. FIG. 1 shows a current approach for planning the insertion path. Three multi-planar reconstruction (MPR) images are separately displayed with a separate volume rendered image. The user scrolls the MPR planes to different locations in order to place the intersection of the planes of the MPR at a point along the path (e.g., center of a vessel). However, scrolling the MPR planes and finding the key points is time consuming, especially for the high resolution volumes with many image slices. Also, the MPR view does not provide a good overview or context of the whole scanned image data.

On the other hand, the user may pick a point on the three-dimensional view. The three-dimensional view is the result of volume rendering, which provides a good overview. However, when the user picks a point on the three-dimensional view, the picked point is defined only in two dimensions and ambiguous along the view direction. The ambiguity results from a given pixel representing many points along a ray in the volume. For example, a user would like to select a point in the middle of the vessel. Depending on the transfer function used for rendering, further error may occur. The transfer function may show the exterior or surface of the vessel, so the picked point is on the surface rather than in the vessel. Other structures may obscure the location for the path in the rendering.

The user can use both 3D and MPR views to navigate the whole data, but this method requires the user to frequently switch among multiple views and takes times. Defining a complex path is time consuming. The desired path may pass through non-homogeneous structures, making consistent identification of the vessel for the path difficult.

### BRIEF SUMMARY

By way of introduction, the preferred embodiments described below include methods, systems, instructions, and computer readable media for path planning using medical imaging. An image rendered from different transfer functions is used. One transfer function provides overall context, such as rendering to show a skin surface or anatomical system. Another transfer function indicates vessel, fluid, or other local information for selecting points along the path. The user may select the transfer function as well as the depth of a clipping plane used at the different local region to allow unambiguous selection of a path point in the three-dimensional rendering.

In a first aspect, a method is provided for planning a path using medical imaging. A first rendering of a volume representing at least a portion of a patient is displayed. The first rendering using a first transfer function. User selection of a second transfer function and a second depth is received. A second rendering is displayed on a second portion of the first rendering. The second rendering is generated with the second transfer function and the second depth. User selection of a second point in the second rendering is received. User selection of a third transfer function and a third depth is received. A third rendering is displayed on a third portion of the first rendering. The third portion is different than the second portion, and the third rendering is generated with the third transfer function and the third depth. User selection of a third point in the third rendering is received. The path for a medical device is defined in the portion of the patient from the second and third points.

In a second aspect, a non-transitory computer readable storage medium has stored therein data representing instructions executable by a programmed processor for planning a path using medical imaging. The storage medium includes instructions for rendering an image with different parts of the image responsive to different transfer functions, indicating first and second parts of the path in the image, the first and second parts responsive to the different transfer functions, respectively, and displaying the path, the path defined by the first and second parts.

In a third aspect, a system is provided for planning a path using medical imaging. A memory is operable to store a dataset representing a three-dimensional volume of a patient. A processor is configured to generate an image from the dataset. The image comprises context responsive to a first transfer function and a path region surrounding the path. The path region is responsive to at least a second transfer function. The processor is configured to define the path in response to selection from the user input on the image. A display is operable to display the image.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 illustrates images used in the prior art for path creation;

FIG. 2 is a flow chart diagram of one embodiment of a method for planning a path using medical imaging;

FIG. 3 is a flow chart diagram of another embodiment of a method for planning a path using medical imaging;

FIG. 4 illustrates an example image resulting from different transfer functions used for planning a path; and

FIG. 5 is a block diagram of one embodiment of a system for planning a path using medical imaging.

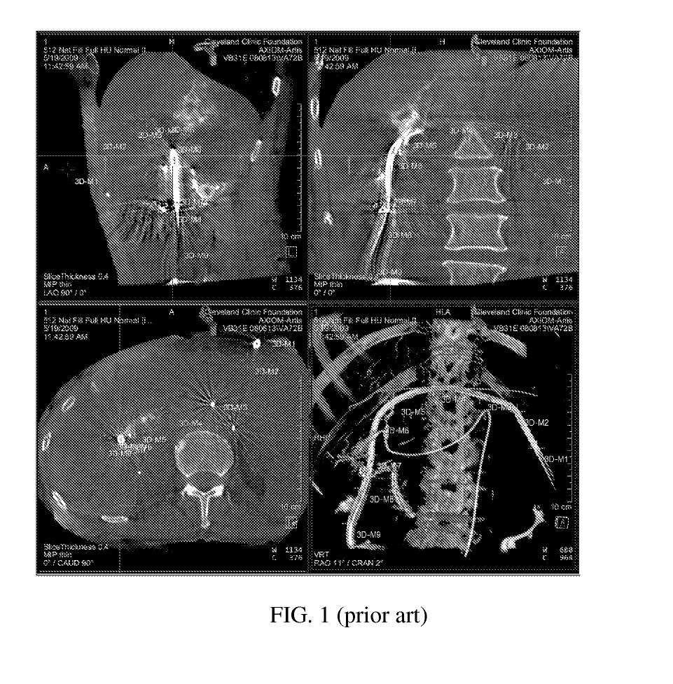

### DETAILED DESCRIPTION OF THE DRAWINGS AND PRESENTLY PREFERRED EMBODIMENTS

Efficient device insertion path definition uses medical imaging. In-context rendering and interaction provides more rapid 3D path creation. Interactive custom intensity classification (e.g., transfer function and/or clipping depth) and picking allows selection of a path point for each of multiple locations, defining a path.

In one embodiment, the path planning is used in an intervention application in the operation room. The insertion path of endoscopic devices, guide wires, or other prosthetic devices is planned. In other embodiments, post-processing software defines a region or path to visualize within the context of a bigger region for analysis.

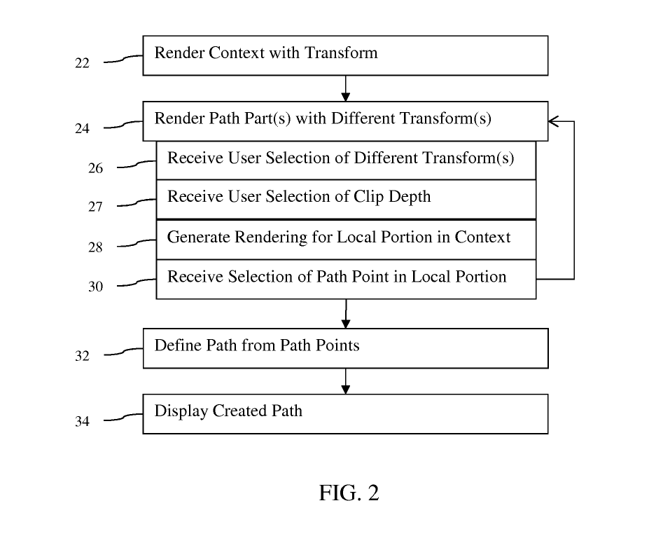

FIG. 2 shows a method for planning a path using medical imaging. The method is implemented by the system of FIG. 5 or another system. In one embodiment, a processor, a graphics processor, or both the processor and graphic processor perform the rendering and defining the path acts. A display may show an image or path rendered by the processor and/or graphic processor. A user interface may be used by an operator to interact with the processor and/or graphics processor for creating the path and/or generating the image.

The acts of the method are performed in the order shown or other orders. For example, the user selection of a transfer function of act 26 may be provided before, during, or after rendering in act 24. A default transfer function for rendering local regions may be provided. In another example, the selection of path points is provided as part of the selection of the different transforms. As the user positions a clipping plane and selects the transform to best or adequately show the anatomy for path selection, the selection of the path point is performed.

Additional, different, or fewer acts may be provided. For example, act 34 is optional. As another example, processor selected or default transforms are used rather than using user selected transforms. In another example, acts associated with adjustment (e.g., window and/or level) of the transform are included.

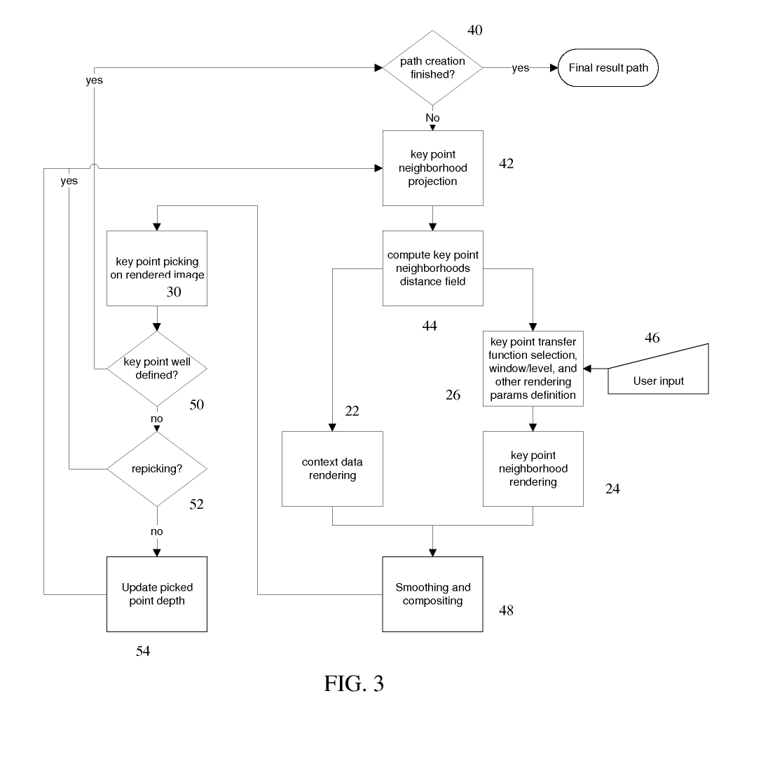

FIG. 3 shows another example of the method. A workflow or process of path creation in a three-dimensional view with different transfer functions selected for context and key point (i.e., path point) neighborhood regions is provided. The workflow of FIG. 3 is implemented by a processor and/or interaction with a processor. The examples of FIGS. 2 and 3 provide two approaches for path definition using different transform functions in a same image. Other approaches may be provided for rendering an image with different parts of the image responsive to different transfer functions.

For rendering, a dataset representing a volume is acquired. The dataset for volume rendering is from any medical modality, such as computed tomography, magnetic resonance, or ultrasound. The dataset is from an actual scan of a patient. Alternatively, part or all of the dataset is artificial or modified. The same dataset is used for rendering with multiple transfer functions.

The dataset is received from a memory, a scanner, or a transfer. The dataset is isotropic or anisotropic. The dataset has voxels spaced along three major axes or other format. The voxels have any shape and size.

Referring to FIG. 2, context is rendered in act 22. The rendering converts the dataset into an image. The volume or three-dimensional region represented by the data is rendered to a two-dimensional image representing the volume as viewed by a virtual person. Volume rendering is performed with the dataset. The rendering application is an application programming interface (API), other application operating with an API, or other application for rendering.

Any now known or later developed volume rendering may be used. For example, projection or surface rendering is used. In projection rendering, alpha blending, average, minimum, maximum, or other functions may provide data for the rendered image along each of a plurality of ray lines or projections through the volume. Different parameters may be used for rendering. For example, the view direction determines the perspective relative to the volume for rendering. Diverging or parallel ray lines may be used for projection. The transfer function for converting luminance or other data into display values may vary depending on the type or desired qualities of the rendering. Sampling rate, sampling variation, irregular volume of interest, and/or clipping may determine data to be used for rendering. Segmentation may determine another portion of the volume to be or not to be rendered. Opacity settings may determine the relative contribution of data along ray lines. Other rendering parameters, such as shading or light sourcing, may alter relative contribution of one datum to other data. The rendering uses the data representing a three-dimensional volume to generate a two-dimensional representation of the volume.

User input or an algorithm defines the desired viewer location and/or other setting of the rendering. The settings are values for rendering parameters, selections of rendering parameters, selections of type of rendering, or other settings. The settings are received as user input, such as an operator selecting a default configuration for a given planning application. Alternatively or additionally, the settings are generated by a processor, such as a processor systematically determining settings based on performance, application, or other criteria. The settings may be predetermined.

In act 22, the rendering is displayed. The rendering represents at least a portion of the patient. For example, the rendering is of a portion for through which the medical device is to travel. For example in cardiac intervention, the portion extends from the upper thigh to the upper chest or other part of the torso. Other portions may be used, such as just the torso, just a leg, including the shoulders or neck, or other parts of the patient. Any continuous range over any axis of the patient may be used. The portion for context includes the likely or possible entirety of the path, but may include more or less. The rendering provides an image for context. By representing the entire volume or that part of the volume through which all or a part of the path to be planned occurs, context for the path is provided.

In one embodiment, an external view of the patient is rendered. A transfer function for emphasizing skin, part of the skin, outer body, fat, or other tissue near a surface of the patient relative to other tissue is used. The emphasis is provided by making the skin or tissue more visible than without emphasis (e.g., intensities from skin tissue are increased relative to intensities from other tissues with or without being the greatest intensities). The rendering may make that external part sufficiently opaque to obscure or cover the internal part of the patient. Alternatively, the external part of the patient is sufficiently transparent to provide representation of both the external part and internal structure of the patient as context. By representing the patient over the portion, more instinctual context for the path is provided to the viewer. The context provides an overall, overview, or generalized perspective on location relative to the patient, but may or may not include tissue specific to the path (e.g., vessel or blood).

In other embodiments, other structure of the patient is emphasized for context instead of the external part. For example, a transfer function for bone is used. As another example, a transfer function for muscle is used. Any anatomical system or part of the patient providing context for the path globally to the patient may be used. Transfer functions for combinations of types of tissue may be used. Depending on the intervention or type of path being planned, different tissue or combinations of tissue may provide context for the path planning.

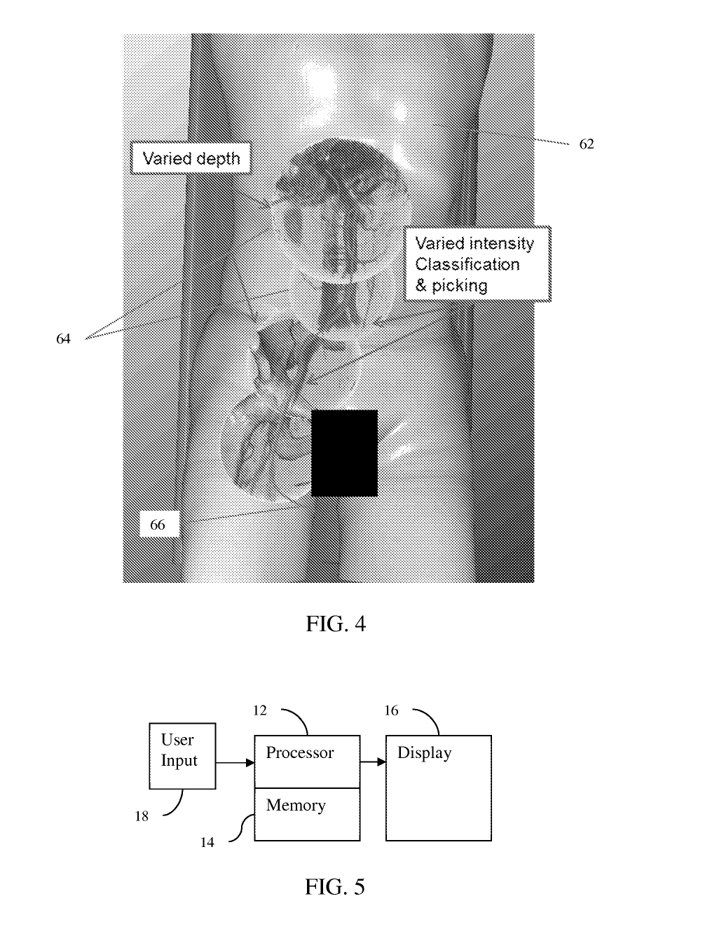

FIG. 4 shows one embodiment of the rendering for context. A skin surface is rendered from the dataset as a context rendering 62. The skin surface may use any type of rendering. Segmentation may be used to render just data from the dataset representing the desired tissue or tissues. The range of the portion of the patient represented is appropriate for a cardiac intervention in this example, so includes the insertion point on the upper thigh through to the heart in the torso.

The context rendering 62 is displayed for assisting the user in creating the path. The context rendering alone may be insufficient for path creation, especially where the context rendering is only of tissue external to the structure immediately surrounding the path to be created (e.g., tissue other than the vessel). Even where a clipping plane is used for the context rendering, the plane may not be at the path depth at all locations.

As shown in FIG. 4, additional renderings using different transfer functions are added to the image in act 24. One or more portions of the volume are rendered with a different transfer function in order to better visualize the vessel, blood, or other indicator of the path. In FIG. 4, path point neighborhood regions 64 are rendered in act 24 (see FIG. 2). The path point neighborhood regions 64 are rendered with relatively translucent transfer functions emphasizing vessels as compared to the relatively opaque transfer function to provide context information. In other embodiments, similar or the same translucency or opposite translucency is used.

Referring to act 24 of FIG. 2, four additional acts 26, 27, 28, and 30 are provided as part of the rendering of act 24. Additional, different, or fewer acts may be used. For example, the selection of act 30 is separate from the rendering, occurring after the rendering is generated in act 28.

In act 24, one or more parts of the patient are rendered for the neighborhood regions 64. For example, one part is initially rendered. The one part may be selected by the user by clicking on the context rendering, assigned by the processor, or otherwise designated. Once a path point or points in the neighborhood region are selected, the neighborhood region 64 is moved by a default amount, user selected amount, to a user selected point on the context rendering, or by a processor selected amount for identifying one or more other points of the path. As another example, multiple neighborhood regions 64 with or without overlap are rendered along the expected path. Segmentation, default based on the application, user input, or other process are used to locate any number of neighborhood regions 64 that are rendered for path selection.

Once the neighborhood region 64 or regions 64 are located, the rendering for one or more of the regions 64 may be altered. Initial renderings may use default settings. Alternatively, the renderings are not performed until one or more settings are received from the user.

In act 26, user selection of a transfer function for one of the neighborhood regions 64 (e.g., currently controlled region) is received. A processor receives the selection from a user interface. The user selects or enters a desired transfer function. For example, the user selects a transfer function from a list of predetermined transfer functions. The list may be in a menu or drop down list. Other user interface structures may be used, such as allowing scrolling through different transfer functions while displaying the results of the currently selected transfer function to the user in the local rendering. The user selects the transfer function most appropriate for the procedure being planned, the location of the neighborhood region 64, and the type of data being rendered (e.g., CT or angiography). The user may vary the selection to find a desired transfer function where several appropriate funtions are available. In alternative embodiments, the processor selects the transfer function based on the application or other criteria.

The transfer function is selected for viewing tissue or fluid for the path. For example, a transfer function emphasizing or configured for viewing blood is selected. As another example, a transfer function emphasizing or configured for viewing vessel walls is selected. In yet another example, a transfer function for viewing both blood and vessel walls is selected. The transfer function may be for mapping gray scale intensities or for mapping colors (e.g., blood flow as red and tissue age brown).

The transfer function selected for the neighborhood region 64 is different than the transfer function for the context. While the rendering for the neighborhood region 64 may provide local context (e.g., tissue adjacent to the vessel or other tissue of interest), the transfer function is different than the one used for the overall context. In the example of FIG. 4, the transfer function for the neighborhood regions 64 excludes or makes more transparent surface tissues (e.g., skin, fat, and/or muscle) as compared to the transfer function for the context rendering 62. Different intensities are emphasized for the neighborhood regions 64 than for the context rendering 62.

The transfer function selected for each of the neighborhood regions 64 is the same or different. For example, different transfer functions may be selected for different neighborhood regions 64. Due to the differences in anatomical location, different transfer functions may be appropriate. For example, bone may obstruct view of the vessel near the hip, so a transfer function more greatly reducing high intensities is selected for a neighborhood region 64 near the hip than for one in the torso. Any consideration may result in a different transfer function for different nieghborhood regions 64. Some of the nieghborhood regions 64 may use the same transfer function.

Other settings may be selected by the user, the processor, and/or adapt to the given planning. For example, shading, lighting, type of rendering or other setting is selected. In one embodiment, the window and/or level for the transfer function is selected. The processor receives alteration or adjustment of the transfer function. For example, the amount of increase or decrease of intensity for emphasis or de-empahsis is altered from one level to another. As another example, the range of intensities or other window function for the transfer function is altered to be broader or narrower.

In act 27, another or alternative setting selected by the user or processor for each neighborhood region 64 is a clip plane depth. The processor receives the position of the clip plane for rendering. The clip plane is a front clip plane where data between the viewer and the clip plane is not used in rendering but data behind the clip plane (i.e., deeper along the view direction) is used. The clip plane is orthogonal to the view direction, parallel to an imaging or rendering plane, or has another orientation. The user controls the depth of the clip plane within the volume, but may alternatively or additionally control the angle or orientation of the clip plane. Similarly, more than one clip plane may be set, such as setting two parallel planes for rendering with data between the planes or setting planes defining lateral boundaries for rendering.

By controlling the clip plane, a distinct point location in a volume may be identified. The clip plane defines a depth. A two-dimensional image rendered with the volume as clipped indicates lateral locations in two-dimensions on the plane. By positioning the clip plane at the depth of the desired path point, the resulting rendering may show the tissue at the path point and tissue adjacent to and behind the point.

In one embodiment, a path planning application predefines different transfer functions, clip plane depth, and other settings that can be used during path creation. Different user interaction may be used to switch among predefined transfer functions, change window/level, update selected point depth, or make other setting changes for one or more neighborhood regions 64 (e.g., selected key or path points on the path). For example, using a mouse wheel to loop or scroll through a predefined transfer function list, using a middle mouse button dragging for window/level changes to the current transfer function, and/or swirling clockwise/counterclockwise on a touch interface to increase/decrease the point depth. As another example, +/− buttons are used to control the depth of the clip plane. Additionally, multiple human input devices maybe used to simultaneously provide the key point location and to select the classification and customer criteria profile (e.g., collection of rendering settings). For instance, using a combination of track ball, joystick and multi-touch displays allows selection of various settings. Furthermore, a combination of settings may be selected from a list of named profiles. Voice control, eye tracking in combination with smart head mounted display devices, or other user inputs may be used for selecting the settings.

In act 28, the neighborhood region 64 is rendered. A portion of the context rendering is rendered using a different transfer function, resulting in a display of two renderings. In the example of FIG. 4, one rendering provides general context and the other rendering provides localized context for path point selection. For path point selection, the rendering is of an internal region of the volume (i.e., internal rendering of the patient).

The neighborhood region 64 for the different rendering is less than ¼ of the volume and/or has a rendered area less then ¼ of the area of the context rendering 62. Other ratios may be provided, such as less than %, less than ⅓, less than ⅕, less than ⅙, or less than ⅛ for the volume rendered and/or the area of the resulting rendering. Each of the neighborhood regions 64 has a same or different size. The size or ratio of sizes is predetermined. Alternatively, the user may set or change the size or ratio.

In the example of FIG. 4, the rendering for the neighborhood is from a hemi-spherical volume. The depth of the clipping plane establishes the flat surface of the hemi-sphere. The data representing locations deeper than the clip plane and within a radius of a center is used for rendering. The center of the circle intersection of the hemi-sphere with the clip plane is set by the user or the processor. The center may correspond to a point for selection, such as the user moving the center until a cross-hair or other target designating the center is at a desired location (e.g., voxel). Alternatively, the center is set separately from any path point selection.

In alternative embodiments, other shapes than a hemi-sphere are used. For example, a cylinder is used where the clip plane provides one end of the cylinder and the imaging plane provides another end. As another example, a cuboid or other volume shape is used. In yet another example, the volume for rendering the neighborhood region is defined based on a cost function. The user selects a seed location. Using thresholding or other calculation, locations of similar, greater, or lower intensity to the seed location are identified and included in the data to be rendered. Other locations are not. Boundaries of tissue structure in the patient may be detected and used to define the extent and/or shape of the neighborhood region 64. A vessel or vessel tree may be located in this way so the a single neighborhood region 64 extends along various candidate vessels for the path.

The location of the neighborhood region 64 is based on user or processor selection. In the image of the renderings, the neighborhood region 64 is shown positioned relative to the anatomy of the context region. The spatial alignment of the voxels of the dataset is used so that the neighborhood region 64 is shown aligned with the context from the context rendering 62. In other embodiments, the neighborhood renderings may be spatially offset. Similarly, the scale of the context and neighborhood renderings is the same, but may be different (e.g., magnifying the neighborhood region relative to the context).

Once the volume extent for the neighborhood region is established, the data is used to render. The volume for the neighborhood region 64 is rendered with the selected classification and interactive depth control. The clipping plane is used to define part of the volume extent. The selected transfer function is applied to data from the volume. As a result, tissues of interest are shown in the resulting rendering. For example, the neighborhood regions 64 are rendered with a transfer function to emphasize the vessel/vein that is interesting to the user for selecting the key points on the path. A different transfer function is used for the neighborhood region 64 than for the context rendering 62. Similarly, the depth setting of the clipping plane results in a different view of the patient for the neighborhood region 64 than for the context rendering 62. The context rendering 62 may or may not have a depth-based clipping plane.

The rendered volume is combined with the context rendering. For example, the rendered volume for the neighborhood region replaces or overwrites any pixels for the context rendering. As another example, the pixels are blended. The blending may occur before or after display value mapping. For gray scale values, the blending may be averaging. For color values, the blending may average color components or a weighted combination of rendered results may be used. In another embodiment, a single rendering is performed, but the different transfer functions are applied as a function of view ray or location. For blending, a combined transfer function is applied. The same data may be applied to both transfer functions and the results used to create a new transfer function. A predetermined combination transfer function may be used. Any interpolation of different transfer functions may be provided.

In one embodiment, the edges or intersection of the neighborhood region 64 with the context rendering is blended, but a center region of the neighborhood region 64 is just rendered using the transfer function selected for the neighborhood region. Any blending, such as averaging, may be used. In one example, the transfer functions from the context rendering and the neighborhood rendering are combined.

Further guidance may be provided using MPR. A graphic representing the neighborhood region 64 may be shown on the images of the MPR assisting the user in placing the neighborhood region.

In act 30, a selection of a path point is received. The selection is a user selection, but may be a processor selection. The user selects a point in the neighborhood region 64. For example, once the neighborhood region 64 is positioned as desired, an activation or selection indication provides for the center of the region 64 on the clip plane to be selected. As another example, the user selects a point in the neighborhood region 64. The selected point may or may not be the center. The point of the path may be selected by mouse clicks, multi-touch display inputs, or any other human interface device interactions. The selection may be updated, such as updating when a user moves the mouse, sliding on a touch interface or clicking with mouse.

The clip plane defines a depth. The point on the image selected by the user is on the clip plane. The position of the selected location in the clip plane in combination with the depth defines a point in three-dimensional space. The user adjusts the clip plane and/or the location on the plane in the neighborhood region 64 to select a point on the path. Similarly, the transfer function is adjusted or selected to better visualize the structure of interest (e.g., blood flow or vessel walls). Once a point in the vessel or other tissue for the path is located, the selection is made.

As represented by the feedback arrow from act 30 to act 24, the rendering and selection may be repeated for other neighborhood regions 64. The repetition may occur once or more, such as at least five, six or tens of times. Each repetition is for selecting a different point along the path. The user may select the number of repetitions or the number may be predetermined. In other embodiments, the number depends on the length of the path where a predetermined spacing is provided between the centers of each neighborhood region 64. By repeating the user selection of the transfer function (act 26), receipt of user selection of the clipping depth (act 27), generation of the rendering of the local portion (act 28), and receipt of user selection of the path point (act 30), multiple points along the path are identified.

Rendering settings for each rendered local neighborhood region 64 are the same or different. For example, a unique classification (e.g., transfer function, depth, window, and/or level) is provided. Since each local region may have different anatomy or internal structure, the user may select settings appropriate for the situation in order to locate a point on the path. The depth is varied to find the point or points on the path for that neighborhood. For example, a custom guidance for an abdomen region may restrict the path point to be within only fluid, gas, or particular type tissue, so the corresponding transfer function is selected.

The user repeats by transitioning sequentially to each neighborhood region 64. Alternatively, each neighborhood region 64 is created when used by the user to pick a point. The user sequentially positions subsequent neighborhoods as desired. Different parts of the path are indicated by selection of the points. Since different situations occur at different locations in the body, different transfer functions, depth, and/or other classification (settings for rendering) may be used for selecting the point. Alternatively, a same transfer function, other settings, and/or depth is used for selection of different points.

As shown in FIG. 4, more than one neighborhood region 64 may be displayed at a same time. In one embodiment, each created neighborhood region 64 is displayed and persists as others are used or created. As a result, some or no overlap between neighborhood regions 64 may result. Any amount of overlap may be provided. For the overlapping portions, the rendered pixels from the more recent or the older rendering may be used. Alternatively, the rendered pixels are blended or the rendering is combined (e.g., a combined transfer function is used). In one embodiment, multiple transfer functions are used to render each pixel from the overlapping regions 64, and the color and opacity values are blended for the pixels inside the overlapping projected regions 64. The blending may be weighted, such as using weights that are a function of the distance from the pixel to the nearest path points or centers of each of the neighborhood regions 64 being blended. In another embodiment, a new transfer function curve is interpolated for a pixel from the transfer function curves of the neighborhood regions 64. Other approaches to provide a smooth or non-smooth transition from one neighborhood region 64 to another and/or the context may be used. In alternative embodiments, only the neighborhood region 64 for which a path point is being picked is displayed on the context rendering 62 at a given time.

In act 32, a path is defined using the selected path points. Since each selected location is defined in three-dimensional space, the specific coordinates of a number of parts of the path are indicated. Any number of points may be defined, such as 3 or more (e.g., tens or hundreds). These points are used to define the path for travel or insertion of the medical device within the portion of the patient represented in the context rendering 62.

In one embodiment, the path is defined by curve fitting. A curve is fit to the points. Any curve fitting may be used, such as interpolation and/or smoothing. A polynomial may be fit to the points. A least squares approach may be used.

In another embodiment, the dataset is used to guide the fitting. The points may be within a particular structure, such as a vessel. The points indicate a specific vessel or vessels for the path as compared to other vessels. Using region growing, segmenting, or other process, the selected vessels or blood flow is found from the dataset. The path is along the center vessel or vessels of the selected path points. By relying, in part, on the dataset, a fewer number of path points may need to be selected by the user as compared to a least squares or other fitting of a curve model. Other approaches for defining the path from selected points may be used.

The defined path may be displayed to the user in act 34. The path may be colored differently, such as shown as green in a gray scale projection. Other highlighting than color may be used, such as brightness, opacity, graphic overlay, and/or magnification. For example, a graphic of the path projected as part of the context rendering 62 is shown. In one embodiment, both local and general context are displayed with the path. The context rendering 62 provides general context relative to the patient. The neighborhood regions 64, such as all or an overlapping sub-set, are rendered and displayed as part of a same image with the context rendering 62. The path 66 is included in the neighborhood regions 64 and highlighted. For example, color is used to show the path. Voxels along the path may be changed to represent a specific color and/or opacity so that the path 66 is emphasized relative to other tissue. In other embodiments, the path 66 is separately rendered and overlaid as part of the same image. A graphic of the path may be created and overlaid.

In another embodiment, the path is used to control rendering along the path. Rather than or in addition to using the neighborhood regions 64, internal tissue within a distance of the path is rendered in one way and the context is rendered in another way (e.g., different transfer functions and/or other classification settings). The result may be showing the path 66 with both internal and external or local and general context. For example, the path curve is projected to the image plane, and a distance field for the projected curve is computed on the image. For any pixels within a given distance of the path in the image plane, a different transfer function than the context rendering 62 is used. Along the path, different transfer functions may have been selected by the user to select specific points. For points of the path in between the user selected points, an interpolated or blended transfer function may be used. A new transfer function is computed for points along the path. Alternatively, the closest transfer function along the path is used. For pixels spaced off of the path but within the threshold distance, the distance field is used to decide which transfer function other than the context transfer function to use. The rendering algorithm computes the nearest point on the path for every pixel, and uses the transfer function for the closest point on the path to render the pixel. Other rendering may be used.

The rendering for showing the path may or may not use clipping. For example, the transfer functions selected to select points on the path may sufficiently result in showing the defined path even with tissue between the viewer and the path being included in the rendering. As another example, the depth of each point along the path is known. A clipping surface extending orthogonally to the view direction at the depths along the path is used. Where the path curves or loops, a shallowest clipping surface may be used.

Different approaches for defining the paths with a combination of transfer functions and/or clipping plane selections are used in FIG. 2. As a result, an interactive method is provided for easy user definition of the path in surgical planning.

FIG. 3 shows another embodiment for selecting the path using different classification for different sub-volumes and/or the overall volume. Additional, different, or fewer acts may be used. For example, act 54 is performed as part of act 26 in looping back to pick a point on the path again, and act 52 is performed as part of act 30. As another example, the distance field of act 44 is not used for smoothing, compositing, or blending, so is not performed.

In act 40, a check is performed for whether the path creation is finished. If not finished, a neighborhood region 64 is initially projection rendered on the image in act 42. The neighborhood region 64 is blended with the context rendering.

In act 44, a distance field is calculated where more than one neighborhood region 64 is provided. The distance field is used to blend any overlapping portions of the neighborhood regions 64.

In act 22, the context is rendered. Using a transfer function to show overall location or location relative to anatomical systems (e.g., skeleton, skin, musculature, vascular, digestive), a context rendering 62 is created. The context rendering and neighborhood region rendering may occur in any sequence with or without user perceptible delay or simultaneously.

In act 26, the user sets and/or varies the transfer function, window, level, and/or clipping depth. The selected classification is entered with user input in act 46. The classification is used to render the neighborhood region 64 in act 24. This rendering may or may not change from the initial rendering in act 42.

The renderings for the neighborhood region 64 and context are shown together as one image. In act 48, the different renderings are smoothed (e.g., filtered) and composited (blended or overlaid). Any filtering may be used. Spatial filtering may be different for the different renderings or may be applied across the entire image.

In act 30, the user selects a point on the path in the clipped neighborhood region 64. In act 50, the user and/or the processor determines whether the selected point is well defined. If the depth or lateral position is off, the point may be picked again in act 52. The depth of the clipping plane may be adjusted for picking again in act 54. If the point is well defined, the process loops back to act 40.

FIG. 5 shows a system for planning a path using medical imaging. The system includes a processor 12, a memory 14, a display 16, and a user input 18. Additional, different, or fewer components may be provided. For example, a network or network connection is provided, such as for networking with a medical imaging network or data archival system.

The system is part of a medical imaging system, such as a diagnostic or therapy ultrasound, x-ray, computed tomography, magnetic resonance, positron emission, or other system. Alternatively, the system is part of an archival and/or image processing system, such as associated with a medical records database workstation or networked imaging system. In other embodiments, the system is a personal computer, such as desktop or laptop, a workstation, a server, a network, or combinations thereof for rendering three-dimensional representations. For example, the system is part of a computer or workstation used by physicians for path planning.

The user input 18 is a keyboard, trackball, mouse, scroll wheel, game pad, joystick, touch screen, knobs, buttons, sliders, touch pad, combinations thereof, or other now known or later developed user input device. The user input 18 generates signals in response to user action, such as user pressing of a button. The signals are received by the processor 12 for controlling rendering of an image and/or path creation.

The user input 18 operates in conjunction with a user interface for user input. Based on a display, the user selects with the user input 18 one or more controls, rendering parameters, path points, depth of a clipping plane, transfer function, window of the transfer function, level of the transfer function, filtering, or other information. For example, the user selects a transfer function from a list, adjusts the depth of the clip plane, and indicates a point on an image. In alternative embodiments, the processor 12 selects or otherwise controls without user input (automatically) or with user confirmation or some input (semi-automatically).

The memory 14 is a graphics processing memory, video random access memory, random access memory, system memory, cache memory, hard drive, optical media, magnetic media, flash drive, buffer, combinations thereof, or other now known or later developed memory device for storing data or video information. The memory 14 is able to store one or more datasets representing a three-dimensional volume of a patient for rendering. Based on configuration by the processor 12 or memory controller, the memory 14 is configured to store and provide access to the dataset.

Any type of data may be used for volume rendering, such as medical image data (e.g., ultrasound, x-ray, computed tomography, magnetic resonance, or positron emission). The rendering is from data distributed in an evenly spaced three-dimensional grid, but may be from data in other formats (e.g., rendering from scan data free of conversion to a Cartesian coordinate format or scan data including data both in a Cartesian coordinate format and acquisition format). The data is voxel data of different volume locations in a volume. The voxels are the same size and shape within the dataset. Voxels with different sizes, shapes, or numbers along one dimension as compared to another dimension may be included in a same dataset, such as is associated with anisotropic medical imaging data. The dataset includes an indication of the spatial positions represented by each voxel. The dataset is stored from a previously performed scan of the patient.

The processor 12 is a central processing unit, control processor, application specific integrated circuit, general processor, field programmable gate array, analog circuit, digital circuit, graphics processing unit, graphics chip, graphics accelerator, accelerator card, combinations thereof, or other now known or later developed device for rendering. The processor 12 is a single device or multiple devices operating in serial, parallel, or separately. The processor 12 may be a main processor of a computer, such as a laptop or desktop computer, may be a processor for handling some tasks in a larger system, such as in an imaging system, or may be a processor designed specifically for rendering. In one embodiment, the processor 12 is, at least in part, a personal computer graphics accelerator card or components, such as manufactured by nVidia, ATI, or Matrox. The processor 12 is configured by software and/or hardware to perform various acts.

The processor 12 is configured to volume render a two-dimensional representation of the volume from the dataset. The two-dimensional representation represents the volume from a given or selected viewing location. Volume rendering is used in a general sense of rendering a representation from data representing a volume. For example, the volume rendering is a projection or surface rendering. The rendering algorithm may be executed efficiently by a graphics processing unit. The processor 12 may be hardware devices for accelerating volume rendering processes, such as using application programming interfaces for three-dimensional texture mapping. Example APIs include OpenGL and DirectX, but other APIs may be used independent of or with the processor 12. The processor 12 is configured for volume rendering based on the API or an application controlling the API. The processor 12 is operable to texture map with alpha blending, minimum projection, maximum projection, surface rendering, or other volume rendering of the data. Other types of volume rendering, such as ray-casting, may be used.

The rendering algorithm renders as a function of rendering parameters. Some example rendering parameters include voxel word size, sampling rate (e.g., selecting samples as part of rendering), interpolation function, size of representation, pre/post classification, classification function, sampling variation (e.g., sampling rate being greater or lesser as a function of location), downsizing of volume (e.g., down sampling data prior to rendering), shading, opacity, minimum value selection, maximum value selection, thresholds, weighting of data or volumes, transfer function, windowing of the transfer function, level of the transfer function, clipping, or any other now known or later developed parameter for rendering. The algorithm may operate with all or any sub-set of rendering parameters. The rendering parameters may be set, fixed, or predetermined. Other rendering parameters may be selectable by the user and/or for rendering a particular dataset. The image is rendered from color data. Alternatively, the image is rendered from grayscale information.

The processor 12 is configured to generate an image from the dataset. The image is rendered using different transfer functions and/or clipping planes for different parts to allow for path planning. The image includes context responsive to a one transfer function and includes a path region responsive to one or more other transfer functions. The path region extends along a possible path or part of the path. In one embodiment, multiple different path regions with or without overlap of each other surround and include respective parts of the path.

The user selects a point on the path using one of the path regions. By selecting or using a transfer function for better distinguishing the path and setting a clipping plane to show the path, the user is able to unambiguously select a point on the path in the rendered path region. As the user makes changes to the rendering and/or makes use of other path regions, the image is updated. A sequence of images rendered from the same dataset is provided. For example, the user may change the transfer function and/or clipping for different path regions, so the image is updated accordingly.

Once the user has selected various path points or while the user is still selecting path points, the processor 12 is configured to define the path. The user selection on the image of points on the path is used to define the path. Curve fitting or other path definition from the points is performed by the processor 12. The user may adjust the path. As or when the path is defined, the image is updated by the processor 12 to show the path.

The display 16 is a monitor, LCD, projector, plasma display, CRT, printer, or other now known or later developed devise for outputting visual information. The display 16 receives images or other information from the processor 12. The received information is provided to the user by the display 16.

The display 16 is part of a user interface. The user interface is for interaction with a user. The user interface may provide selectable options for rendering and/or selection of path points.

The memory 14 and/or another memory stores instructions for operating the processor 12. The instructions are for path planning using medical imaging. The instructions for implementing the processes, methods, and/or techniques discussed herein are provided on non-transitory computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for planning a path using medical imaging, the method comprising:
displaying a first rendering of a volume representing at least a portion of a patient, the first rendering using a first transfer function;
receiving user selection of a second transfer function and a second depth;
generating a second rendering displayed on a second portion of the first rendering, the second rendering being generated with the second transfer function and the second depth;
receiving user selection of a second point in the second rendering;
receiving user selection of a third transfer function and a third depth;
generating a third rendering displayed on a third portion of the first rendering, the third portion different than the second portion, the third rendering being generated with the third transfer function and the third depth;
receiving user selection of a third point in the third rendering; and
defining the path for a medical device in the portion of the patient from the second and third points.

2. The method of claim 1 wherein displaying comprises displaying the first rendering of at least part of a thigh and part of a torso of the patient.

3. The method of claim 1 wherein displaying comprises displaying the first rendering where the first transfer function emphasizes skin relative to internal structure.

4. The method of claim 1 wherein receiving the user selection of the second transfer function comprises receiving a different transfer function for the second rendering than the first transfer function.

5. The method of claim 1 wherein receiving the user selection of the second depth comprises receiving a position of a clipping plane for the second rendering, and wherein generating the second rendering comprises generating the second rendering with data of the volume deeper than the clipping plane along a view direction.

6. The method of claim 1 wherein generating the second rendering comprises generating the second rendering for an internal region of the volume, the internal region comprising less than ¼ of the volume and the second portion being less than ¼ of an area of the first rendering.

7. The method of claim 1 wherein generating the second rendering comprises generating the second rendering with the second portion being anatomically aligned with the first rendering.

8. The method of claim 1 wherein receiving user selection of the second point comprises determining the second point in the volume based on the second depth and a location in the second rendering.

9. The method of claim 1 wherein receiving user selection of the second and third transfer functions comprises receiving user selection of different transfer functions, and wherein the third portion is different but overlaps with the second portion.

10. The method of claim 1 wherein receiving user selection of the second and third depths comprises receiving user selection of different depths, and wherein the third portion is different but overlaps with the second portion.

11. The method of claim 1 further comprising repeating the receiving of transfer function and depth, generating a rendering, and receiving point selection from the rendering for each of fourth, fifth, and sixth portions.

12. The method of claim 1 wherein defining the path comprises fitting a curve to the second and third points.

13. The method of claim 1 further comprising receiving user selection of a window, level, or window and level with the second transfer function.

14. In a non-transitory computer readable storage medium having stored therein data representing instructions executable by a programmed processor for planning a path using medical imaging, the storage medium comprising instructions for:
rendering an image with different parts of the image responsive to different transfer functions;
indicating first and second parts of the path in the image, the first and second parts responsive to the different transfer functions, respectively; and
displaying the path, the path defined by the first and second parts.

15. The non-transitory computer readable storage medium of claim 14 wherein rendering the image comprises rendering from data for a portion of a patient and rendering the first and second parts as different regions of the portion, and wherein indicating the first and second parts comprises receiving user selection of first and second points.

16. The non-transitory computer readable storage medium of claim 15 wherein rendering from the data for the portion comprises rendering an external view of the portion, and wherein rendering the first and second parts comprises rendering internal views of the different regions.

17. The non-transitory computer readable storage medium of claim 14 wherein rendering the different parts comprises rendering as a function of the different transfer functions and different depths, the different transfer functions and depths being user selected.

18. The non-transitory computer readable storage medium of claim 14 wherein displaying the path comprises highlighting a curve fitted to the first and second parts on the image.

19. A system for planning a path using medical imaging, the system comprising:
a memory operable to store a dataset representing a three-dimensional volume of a patient;
a user input;
a processor configured to generate an image from the dataset, the image comprising context responsive to a first transfer function and comprising a path region surrounding the path, the path region responsive to at least a second transfer function, and the processor configured to define the path in response to selection from the user input on the image; and
a display operable to display the image.

20. The system of claim 19 wherein the processor is configured to generate the image as a sequence of images where user input selects the second transfer function and a third transfer function for different parts of the path region and selects depths for the different parts of the path region, and wherein the processor is configured to define the path in response to selections of points at the depths in the different parts.
